(19) United States
(12) Patent Application Publication
(10) Pub. No.: US 2012/0100598 A1
(43) Pub. Date: Apr. 26, 2012

(54) R. rhodochrous and Linoleic Acid As Bioremediation Agents of Polycyclic Aromatic Hydrocarbons (PAH) in Diesel-Contaminated Soil

(76) Inventor: Morgan Walker Sinko, San Antonio, TX (US)

(21) Appl. No.: 13/252,318

(22) Filed: Oct. 4, 2011

Related U.S. Application Data
(60) Provisional application No. 61/406,187, filed on Oct. 25, 2010.

(51) Int. Cl. A62D 3/02; C02F 3/00; C12S 99/00; C12N 1/20; C02F 101/32

(52) U.S. Cl. 435/262.5; 435/252.1; 210/601

(57) ABSTRACT

Different bioremediation agents' effectiveness against concentrations of diesel fuel in soil were tested. The purpose of this experiment is to find a quick, clean, and cost-effective alternative to current remediation techniques using new bioremediation techniques. Analysis indicated that the use of a mixture of Linoleic acid and R. rhodochrous generated the greatest, quickest, and longest lasting degradation rate. The mixture provided a 51.23% drop in PAH and data suggests that it will to remediate after the end of the 4th week. In second place came the Linoleic acid with an immediate drop of 36.61% before leveling out in the 2nd week. The bacteria (R. rhodochrous) started slow but moved into a greater rate every week bringing it to an overall 46.65% change. The hypothesis that the mixture of the R. rhodochrous and Linoleic acid would yield the greatest PAH degradation was supported by the data.

### CROSS REFERENCE TO RELATED APPLICATIONS

This application claims priority from U.S. Patent Application Ser. No. 61/406,187, entitled “R. rhodochrous and Linoleic Acid As Bioremediation Agents of Polycyclic Aromatic Hydrocarbons (PAH) in Diesel-Contaminated Soil”, filed on 25 Oct. 2010. The benefit under 35 USC §119(e) of the United States provisional application is hereby claimed, and the aforementioned application is hereby incorporated herein by reference.

### FEDERALLY SPONSORED RESEARCH

Not Applicable

### SEQUENCE LISTING OR PROGRAM

Not Applicable

### TECHNICAL FIELD OF THE INVENTION

The present invention relates generally to the degradation of contaminates. More specifically, the present invention relates to the bioremediation of contaminates of Polycyclic Aromatic Hydrocarbons (PAH) in Diesel-Contaminated Soil.

### BACKGROUND OF THE INVENTION

One of the most dangerous contaminates in our world is right under our feet. Polycyclic Aromatic Hydrocarbons, otherwise known as PAH, are a collection of hundreds of organic chemicals that are extremely carcinogenic and mutagenic. PAH are the second most dangerous contaminates in the world and the third leading cause of cancer in the United States. Until recently, attempts at remediation of PAH from natural and unnatural sources have been costly, complicated and have had mixed results. The rising number of PAH sources makes PAH increasingly difficult to remove from urban and more rural environments.

Polycyclic Aromatic Hydrocarbons (PAH), a byproduct of incomplete combustion, is a serious global environmental problem. The carcinogenic/mutagenic effects of PAH were first discovered after WWII when gas plumes from manufacturing plants in England resulted in the near destruction of their fishing industry. Several years and millions of dollars were spent on chemical remediation but few advances in these techniques have occurred in the last 60 years.

PAH come from many sources both natural (biogenic) and unnatural or manmade (anthrogenic). PAH is generated by the incomplete combustion of carbon-based substances such as wood, oil, coal, tar and gasoline. The top contaminate source of PAH is the exhaust of automobiles. PAH travel in the air and eventually settle into soil. PAH can leach into waterways and choke out entire ecosystems, threatening both the environment and humans. Aquifers and swamps are in danger. Ingestion or inhalation of PAH can cause blindness, hair loss, internal hemorrhaging, lung cancer and birth defects, making city dwellers particularly vulnerable. PAHs are a normal part of life, but with current industrialization, and the increased number of cars on the road today, PAH levels are at an all-time high.

PAH are classified by their molecular weight, or the number of carbon rings in their structure. There are multiple types of PAH that we come in contact with on a daily basis. We often come into contact with high molecular weight (HMW) PAH such as benzo[a]pyrene that contain five rings and are particularly carcinogenic. PAH with very high molecular weight are dangerous, but are too large be absorbed by the body and are simply excreted out. Low molecular weight (LMW) PAH contain 1-3 molecular rings and are hazardous and small enough to be absorbed into your bloodstream during every day activities. The most common form of natural PAH is LWM Naphthalene. Sixteen extremely carcinogenic PAH are targeted and monitored by the Environmental Protection Agency and members of the European Union and are commonly known as “target” PAH.

Governments around the world have begun to take action to prevent and reverse PAH pollution. For example, in the late 1940's Britain conducted an unprecedented clean up of the oceans surrounding the factories on its West Coast, which were so contaminated that the fish populations were drastically depleted. The chemicals used were so expensive that the government was forced to use funds that could have been used for post-war reconstruction, but people were starving due to the lack of fish. In late 2007, the Chinese government ordered the first attempted large-scale PAH cleanup of the lakes and swamps around Hong Kong using bacteria that feed on these organic chemicals, similar to the function of nematodes. Germany requires that all household products be tested for PAH levels before they are put on the market.

Bacteria have been found that reduce PAH. Unfortunately, Inventors have found that after introducing R. rhodochrous, and Sphingomonas to heavily contaminated swampland, they did not reduce the levels of PAH fast enough to be immediately effective. But these bacteria could serve as a slow acting, long-term solution to the world's PAH conundrum. R. rhodochrous is unique among other mycobacterium, in that it is benign to humans, and that it excretes lipids to cover its surface. PAH are fat-soluble due to a non-polar structure, so as the bacteria comes into contact with more PAH, more fatty lipids are excreted dissolving the PAH and then converting it into food for R. rhodochrous. Afterward the R. rhodochrous transforms the now homogeneously mixed PAH/lipids into water, biodiesel, and naturally occurring steroids.

In other studies, strawberries were planted in batches of spiked soil to identify the capability of naturally secreted Linoleic acid to degrade PAH. Linoleic acid research is rare and has not gone through large enough scale testing. (i.e. deep soil testing, unfavorable growing conditions in polluted areas, etc.). Linoleic acid can be found in all plants, fruits, and vegetables but especially in potatoes, strawberries, safflowers, carrots, and even radishes. The safflower oil used in this experiment is 76.7% Linoleic acid.

Linoleic acid is a fatty acid form of lipids. PAH (which are fat soluble due to nonpolarization) cannot dissolve in water, but can dissolve in lipids. The Linoleic acid does so by being oxidized by the PAH. The downside of Linoleic acid is that one of the byproducts is the known carcinogen 4-Hydroxynonenal (4-HNE). Fortunately 4-HNE is consumed by R. rhodochrous because of its chemical structure being similar to the food created by R. rhodochrous' lipid shield.

Inventors all over the world have taken strides toward PAH prevention and protection. The use of purely electric cars is a way to cut off the source of PAH, but still leaves us to worry about current PAH levels. MGP (Manufacturing Gas Plants) are areas that were used in the 19th and 20th century powered by coal, which produced gas plumes that have rendered areas (usually in prime real estate) completely unlivable. The land is devoid of life and the air inside and around the abandoned facilities is nearly toxic. As a result of this unchecked industrialization, PAH has choked the life out of these formerly pristine areas.

### SUMMARY OF THE INVENTION

The inventor performed an experiment to determine the effect of distance from the source on PAH concentrations in roadside soil. The data revealed that PAH concentrations in soils decrease as sample sites get farther from the source. The experiment tested for heavy, total, and target (hazardous) PAH. The inventor found that dangerous levels of anthrogenic PAH in roadside soil that decreased at an average rate of 0.54 ppm/10 ft. The inventor's early experimentation proved that as you got farther from the source of a PAH emitter (cars), the levels decreased.

5, Findings such as these have lead scientists and engineers to look toward bioremediation as a way of fixing the PAH problems. Bioremediation is the process of returning nature that has been contaminated to its original condition through the use of natural materials such as bacteria or plants. Inventors have used bacteria and other bio sources such as wheat or plants containing Linoleic acid to degrade PAH.

Using the new TD-500 and UVF-3100 PAH testing kits different bioremediation agents' effectiveness against concentrations of diesel fuel in soil were tested. The purpose of this experiment is to find a quick, clean, and cost-effective alternative to current remediation techniques using new bioremediation techniques. The hypothesis is that the R. rhodochrous/Linoleic acid mixture will yield the highest decrease in PAH concentration. The null hypothesis is that the R. rhodochrous/Linoleic acid mixture will have no effect on the PAH concentration.

Four containers of soil were spiked with diesel fuel, a PAH source, to a concentration of 1,000 ppm. Container 1 (control) had no additions, while 1.575 ml of Linoleic acid was added to container 2, and R. rhodochrous to container 3. A combination of the agents was added to container 4.

Three samples were collected from each container weekly for four weeks and tested for PAH concentration using UV fluorescence spectroscopy. The Linoleic acid/R. rhodochrous mixture resulted in the highest degradation (51.23%) of PAH followed by R. rhodochrous alone with 46.65% degradation, and Linoleic acid alone with an initial 36.61% remediation before leveling out. PAH concentration in the control remained relatively static. This implies that R. rhodochrous metabolized the PAH. Future research will be conducted over longer periods with varying soil types and temperatures to determine the best applications for these agents.

Therefore it is an objective of the present invention to develop a quick, clean, and inexpensive bioremediation solution to PAH pollution. It was hypothesized that a combination of Linoleic acid and Rhodococcus rhodochrous (R. rhodochrous); would yield the greatest degradation of PAH. The hypothesis that the mixture of the R. rhodochrous and Linoleic acid would yield the greatest PAH degradation was supported by the data.

### BRIEF DESCRIPTION OF THE DRAWINGS

The accompanying drawings, which are incorporated herein an form a part of the specification, illustrate the present invention and, together with the description, further serve to explain the principles of the invention and to enable a person skilled in the pertinent art to make and use the invention.

FIG. 1 depicts the percent decrease in PAH concentrations produced by each of the three bioremediation agents and in the control;

FIG. 2 shows the levels of PAH over the four weeks when no bioremediation agents were introduced into the container;

FIG. 3 represents the PAH concentrations in the container that was introduced to safflower oil, also known as Linoleic acid;

FIG. 4 portrays the effects of R. rhodochrous bacterium after it had been introduced to a diesel-spiked container;

FIG. 5 shows the effects when a mixture of both R. rhodochrous and safflower oil (Linoleic acid) are introduced to a container of diesel-spiked soil;

FIG. 6 depicts the Concentration of Diesel over Time;

FIG. 7 depicts the Percentage of Oleic acid over Time;

FIG. 8 is Red Clay Chromatograms;

FIG. 9 is Limestone Chromatograms;

FIG. 10 is Sand Chromatograms;

FIG. 11 is Topsoil Chromatograms; and

FIG. 12 is Contractor Mix Chromatograms.

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus of the present invention.

Polycyclic aromatic hydrocarbons (PAH) are a collection of chemical compounds, which are created from the incomplete combustion of carbon-based materials. PAH are extremely carcinogenic, and can lead to (among other things) kidney failure, internal hemorrhaging, and death. Over the last two years the Inventor has studied PAH and means of remediating them. In 2008, the Inventor performed an experiment to determine the effect of distance from the source on PAH concentrations in roadside soil. The data revealed that PAH concentrations in soils decrease as sample sites get farther from the source. The experiment tested for heavy, total, and target (hazardous) PAH. The Inventor found that dangerous levels of anthrogenic PAH in roadside soil decreased at an average rate of 0.54 ppm/10 ft. The Inventor proved that as you got farther from the source of a PAH emitter (cars), the levels decreased.

The present invention is a Linoleic acid/Rhodococcus rhodochrous mixture designed to develop a reliable and economically feasible bioremediation method against PAH. The present invention, discovered through experimentation by the Inventor, proved that bioremediation could be equally or more effective than current remediation techniques. After two months of remediation there was a 97% decrease of PAH in the treated soil.

The ratio of the Linoleic acid to the R. rhodochrous bacteria believed to be the best mode of the present invention is three parts Linoleic acid to one part R. rhodochrous bacteria. A ratio wherein the bioremediation mixture includes from about 5% to about 50% by weight of the natural microorganism source and a corresponding amount of about 5% to 50% by weight of the Linoleic acid may also be used in alternative embodiments.

Linoleic acid was chosen on the basis that it is cost efficient and can be found in abundance at any health food store. Linoleic acid is a polyunsaturated fatty acid, which goes through a free radical reaction to create extra food for the bacteria. Linoleic acid has been tested as a PAH bioremediation tool and was rejected due to a hazardous byproduct known as 4-hydroxynonenal (4-NHE). 4-HNE is very volatile and hazardous to human health. However, once coupled with the R. rhodochrous the 4-NHE is metabolized and replaced with safe water and methyl esters.

R. rhodochrous was chosen on the basis that it is stable and safe. It is not considered a pathogen and is one of the only bacteria that can effectively metabolize the PAH, and does not cause tuberculosis. R. rhodochrous is also native to a large portion of North America allowing it to survive the temperature of many distinct regions. R. rhodochrous also has many unique devices at its disposal to protect itself from the toxicity of PAH and other diesel additives. R. rhodochrous is capable of creating a lipid coating around itself when it comes into contact with PAH, acting similar to a shield. This reaction yields chemicals similar to 4-NHE that are then metabolized into water and methyl esters. Thus the synergy of Linoleic acid and R. rhodochrous remediates the diesel with unprecedented results. The 5 different soil types were chosen based on the variety of environments that the bioremediation agent might be deployed in, from the sands of Saudi Arabia to the clay landscapes of Arizona.

Now referring to the Figures, the purpose of the study, and the best mode of the present invention, was to develop a quick, clean, and inexpensive bioremediation solution to PAH pollution. It was hypothesized that a combination of Linoleic acid and Rhodococcus rhodochrous (R. rhodochrous); would yield the greatest degradation of PAH.

Prior to the experiment, R. rhodochrous was grown in a Petri dish. When the bacterium was prepared, 757.5 g of soil were loaded into each of four 8-liter containers. The containers were labeled “Control,” “Bacteria,” “Linoleic Acid,” and “Mixture” respectively. 2.755 ml of diesel fuel were then stirred into the soil for five minutes to ensure even dispersal and the release of all benzene gasses from the diesel.

Four containers of soil were spiked with diesel fuel, a PAH source, to a concentration of 1,000 ppm. Container 1 (control) had no additions, while 1.575 ml of Linoleic acid was added to container 2, and R. rhodochrous to container 3. A combination of the agents was added to container 4.

In the tank labeled “Bacteria” the R. rhodochrous was transferred from the Petri dish to the tank, distributing the microbes as evenly as possible across the surface of the soil. In the container labeled “Linoleic Acid,” 1.575 ml of safflower oil were distributed evenly across the soil. In the tank labeled “Mixture,” 1.575 ml of safflower oil were distributed evenly across the soil and then a layer of R. rhodochrous microbes was placed on top of the soil. No bioremediation agent was introduced into the “Control” container.

Three soil samples were taken from each container over a four-week period on days 0, 7, 14, and 21 and 28. The first sample taken on each day was from the bottom of the container, the second was from the center, and the third was from the surface. The samples were tested in a TD-500 ultraviolet analyzer according to the standard user guide and the results were recorded. The Linoleic acid/R. rhodochrous mixture resulted in the highest degradation (51.23%) of PAH followed by R. rhodochrous alone with 46.65% degradation, and Linoleic acid alone with an initial 36.61 remediation before leveling out. PAH concentration in the control remained relatively static. This implies that R. rhodochrous metabolized the PAH. Future research will be conducted over longer periods with varying soil types and temperatures to determine the best applications for these agents.

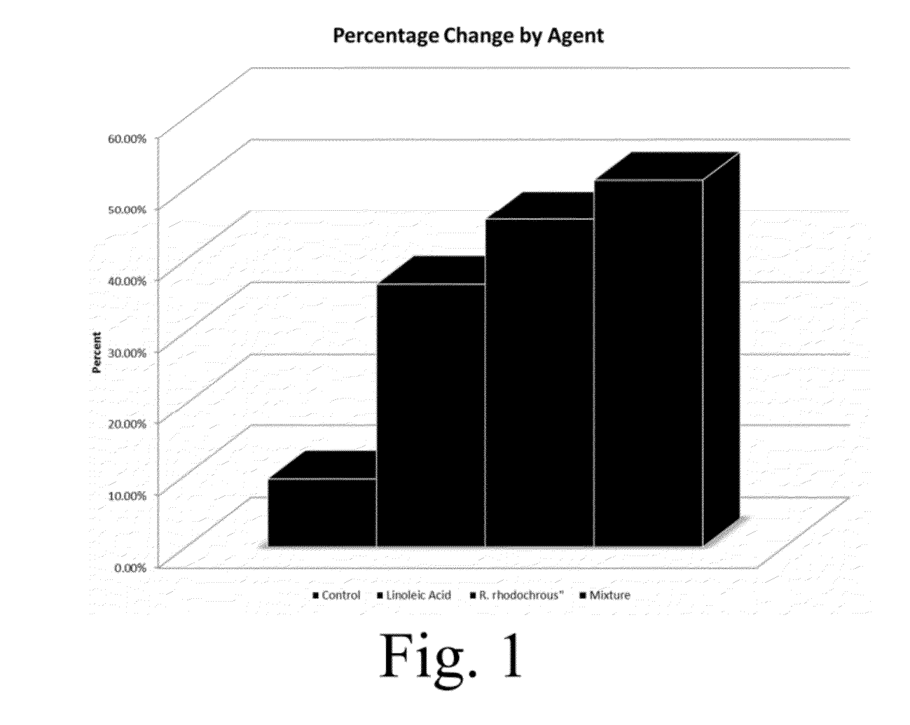

FIG. 1 depicts the percent decrease in PAH concentrations produced by each of the three bioremediation agents and in the control. The Linoleic acid/R. rhodochrous mixture proved 4.58% more effective on remediating PAH then the R. rhodochrous alone and 14.62% more effective than the Linoleic acid alone.

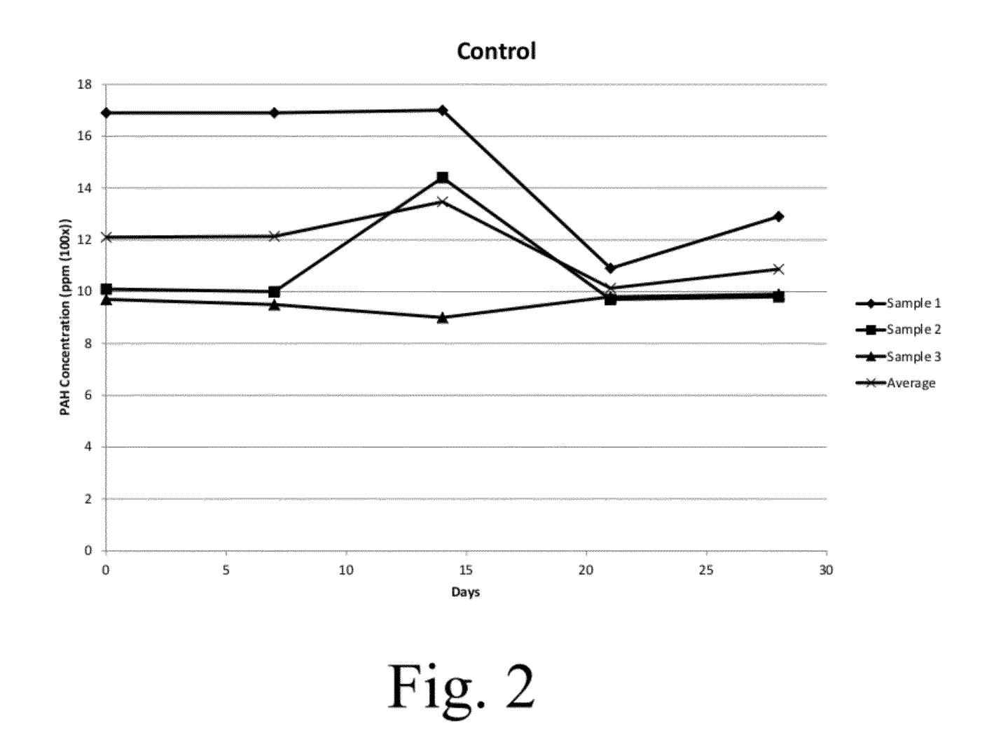

FIG. 2 shows the levels of PAH over the four weeks when no bioremediation agents were introduced into the container. With nothing to counteract the diesel, the concentration of PAH remained at around 1200 parts per million. The overall change was a negligible 9.4 percent, which is the result of improper proportions during the second to last test of sample I.

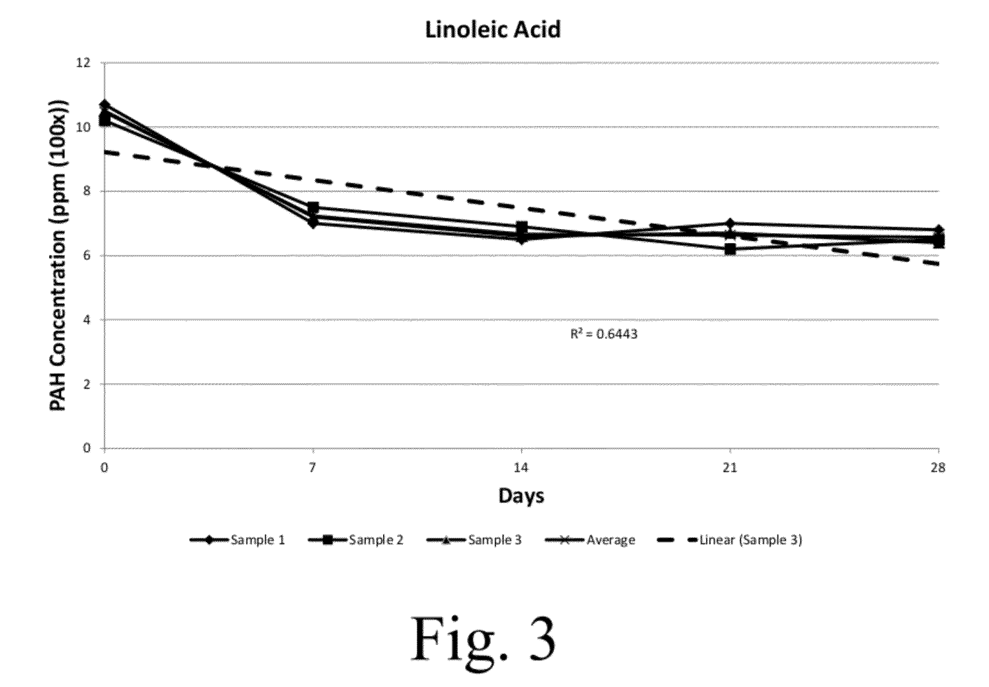

Now referring to FIG. 3, the graph represents the PAH concentrations in the container that was introduced to safflower oil, also known as Linoleic acid. Then in the first week Linoleic acid yielded a greater drop than any other agent. The overall change from day 0 to day 28 was 36.61 percent. Based on the “leveling out” of the graph is not likely that the acid would yield further results. Further results could be produced if plants containing Linoleic acid were grown in the soil.

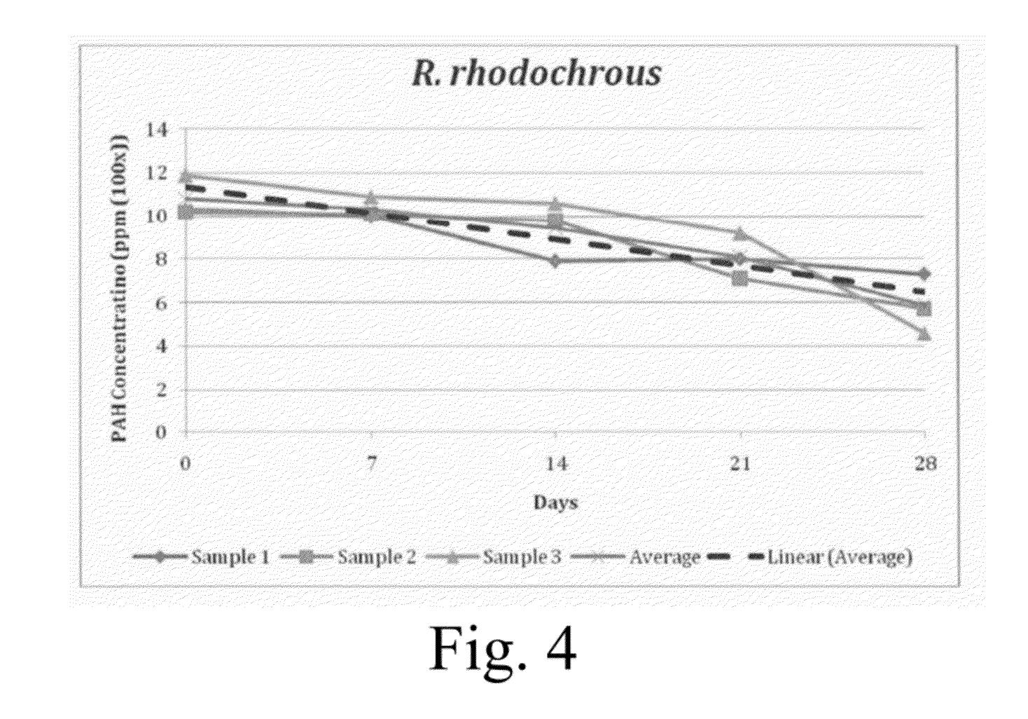

FIG. 4 portrays the effects of R. rhodochrous bacterium after it had been introduced to a diesel-spiked container. During the first week the degradation rate of the PAH seemed negligible, but immediately afterward the rate increased. This most likely showed a growth in the amount of bacteria. Week 3 (day 14-21) showed the greatest jump, and will continue to show results as a more long-term solution. Over the 3 weeks the R. rhodochrous showed an average 46.65% degradation.

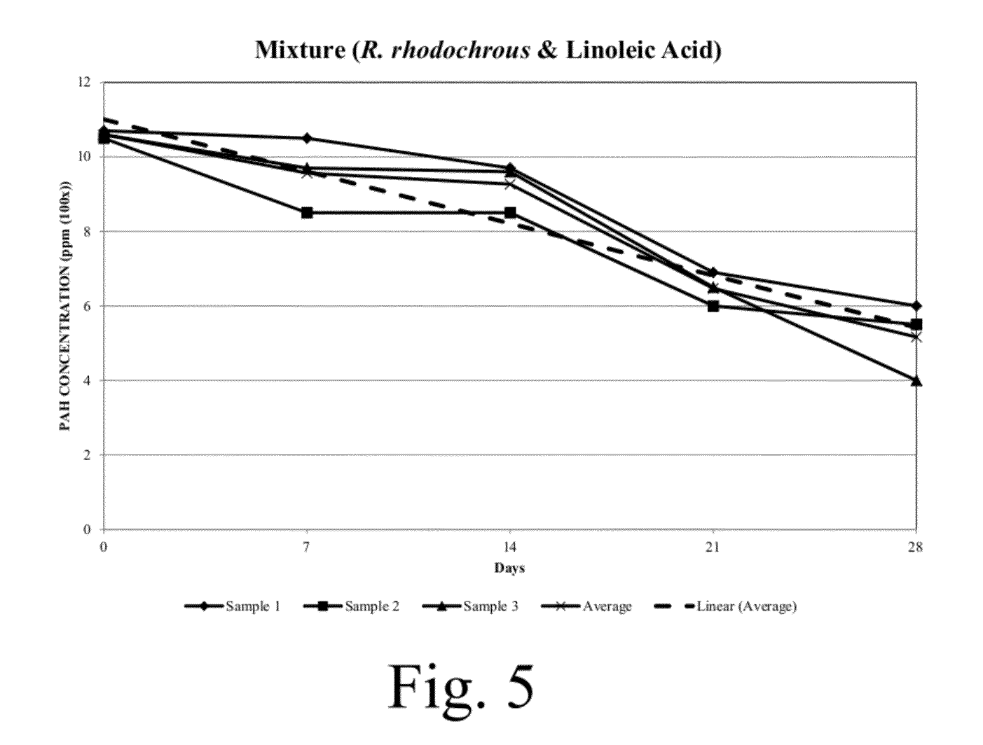

Now referring to FIG. 5, the graph shows the effects when a mixture of both R. rhodochrous and safflower oil (Linoleic acid) are introduced to a container of diesel-spiked soil. The first week showed excellent result similar to that of the Linoleic acid container. But then switching to the trend of bacteria and using the acid as a lipid auger, the bacteria showed an almost exponential degradation rate per week. The mixture technique will most likely be a viable option for fast acting and long term relief. The overall percentage was the greatest of the containers with a whopping 51.23%.

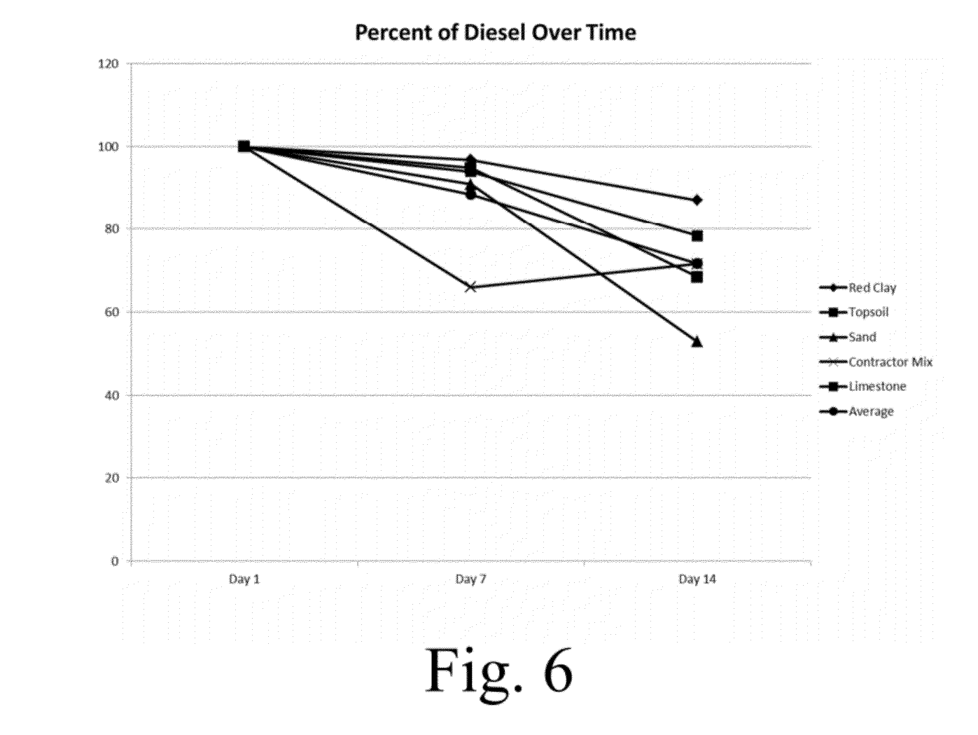

FIG. 6 depicts the percent decrease in PAH concentrations produced by each of the three bioremediation agents and in the control. The Linoleic acid/R. rhodochrous mixture proved 4.58% more effective on remediating PAH then the R. rhodochrous alone and 14.62% more effective than the Linoleic acid alone.

Table 1 portrays the levels of every sample along with the average per week. The average change per week is easily available by comparing the shaded areas for every column. The Linoleic made the quickest and most abrupt drop from 10.47 to 7.23 ppm ((100×) 3.24 drop)). The second week's largest drop was produced by the R. rhodochrous container, which dropped from 10.33 to 9.43, which is equivalent to a 9% degradation. The third week's most profound drop of 9.27 to 6.47 was prevalent in the R. rhodochrous/Linoleic acid mixture with a difference of 2.9 ppm. The fourth and final weeks largest drop was produced by the R. rhodochrous from 8.10 to 5.87 (−2.23 change).

TABLE 1
The concentration levels of every sample along with the per week average.

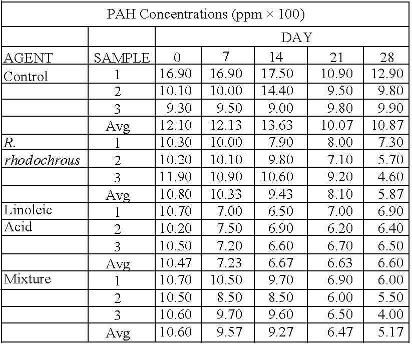

Analysis indicated that the use of a mixture of Linoleic acid and R. rhodochrous generated the greatest, quickest, and longest lasting degradation rate. The mixture provided a 51.23% drop in PAH and data suggests that it will to remediate after the end of the 4th week. In second place came the Linoleic acid with an immediate drop of 36.61% before leveling out in the 2nd week.

Finally the bacteria (R. rhodochrous) started slow but moved into a greater rate every week bringing it to an overall 46.65% change. The hypothesis that the mixture of the R. rhodochrous and Linoleic acid would yield the greatest PAH degradation was supported by the data.

In a second experiment, the effectiveness of a Linoleic acid/R. rhodochrous mixture as a bioremediation agent of Polycyclic Aromatic Hydrocarbons (PAH) in a real-world, uncontrolled setting. Ten tanks, each two containing one of 5 different soil types, were spiked with diesel fuel. One of each soil type was introduced to the Linoleic Acid/R. rhodochrous mixture. Three samples were taken from each tank every 7 days. Each of the three samples was homogenized, prepared, and then analyzed through a gas-chromatograph-mass-spectrometer using standard EPA Method 8207C.

Although originally targeting PAH specifically, the bioremediation mixture in reality converts the entirety of diesel fuel into water as well as environmentally friendly methyl esters, the main ingredient of bio-fuel. The overall diesel concentration decreased by an average decrease of 29.45%. The most efficient soil was the sand, with an overall remediation of 53%. Future studies using pure controlled compounds, and re-inoculating the soil at set intervals, as well as simulated oil spill cleanup using the agents are also being investigated. These results show that the R. rhodochrous/Linoleic acid mixture has the capability to help construct a sustainable planet in a fossil fuel dominated world.

Prior to the beginning of the experiment, five Petri dishes of R. rhodochrous were grown for 7 days. Ten 1.133 m containers were laid outside and marked to identify which soil they would hold. Two containers each were filled with crushed limestone, sand, red clay, contractor mix, and topsoil. Each container held about 200 kg of soil. Every container was spiked with diesel fuel to a concentration of 2000 ppm by weight.

One container of each soil type was treated with the Linoleic acid/R. rhodochrous mixture. Soil temperatures were taken every night at 7:00 pm and daily high/low air temperatures were recorded to document the environmental conditions impacting the bioremediation mixture. Three samples were taken from each container on days 1, 7, and 14. The three samples were homogenized (to create an average) and prepared through the extraction process for a GC-Mass Spectrometer standard EPA Method 8207C. Results were gathered and analyzed.

FIG. 6 represents the overall drop in diesel percentage by soil type over time. The lower the percentage, the more diesel fuel has been converted into water and methyl esters. While red clay and topsoil show the least remediation, their bioremediation rate closely resembles data from previous samples in an earlier lab experiment that were treated only with R. rhodochrous. This finding suggests that the denser soils absorb the Linoleic acid slower than the less compact soils, retarding the availability of the growth medium to the bacteria. Less compact soils such as the sand absorbed the Linoleic acid more quickly resulting in faster remediation. FIG. 6 indicates slight remediation in the first week followed by constantly increasing amounts of remediation in later weeks. The average remediation of diesel across all soil types after two weeks was 29.9%.

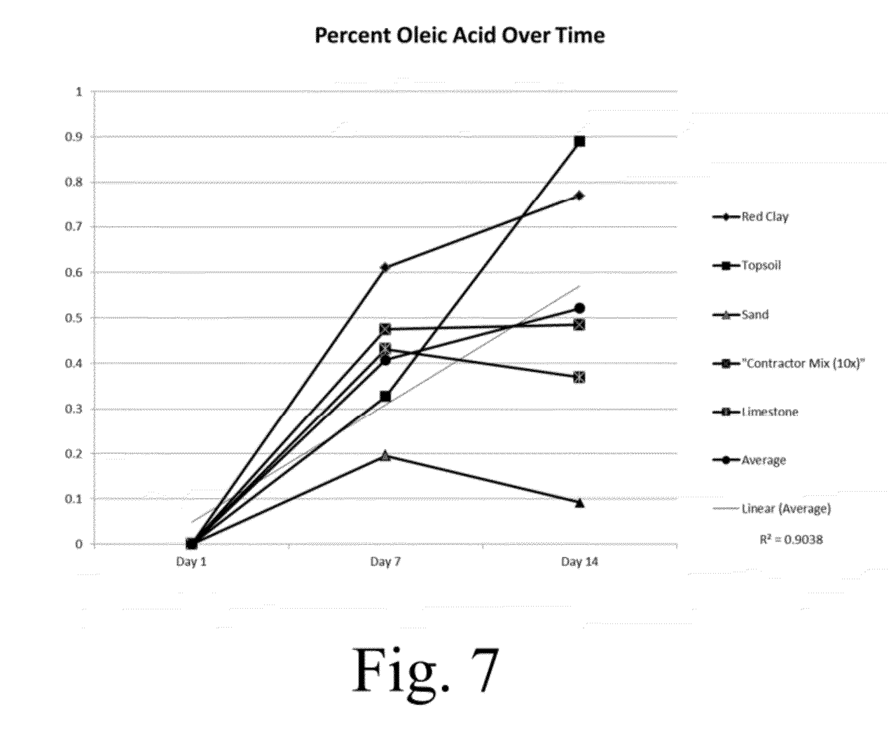

FIG. 7 depicts the percent of oleic acid by soil type over time. Oleic acid is the metabolite of the bacteria. An increase in the metabolite suggests an increase in bacterial activity in the soil. FIG. 7 represents oleic acid as a percentage of the overall sample. While the percentages appear small, they are relatively large within the sample and consistent with other diesel compounds. The contractor mix in FIG. 7 is shown at a 1/10 scale in order to give detail to the graph. The contractor mix's unusually high levels of oleic acid are most likely due to the mixture's reactions with other compounds found in the soil itself.

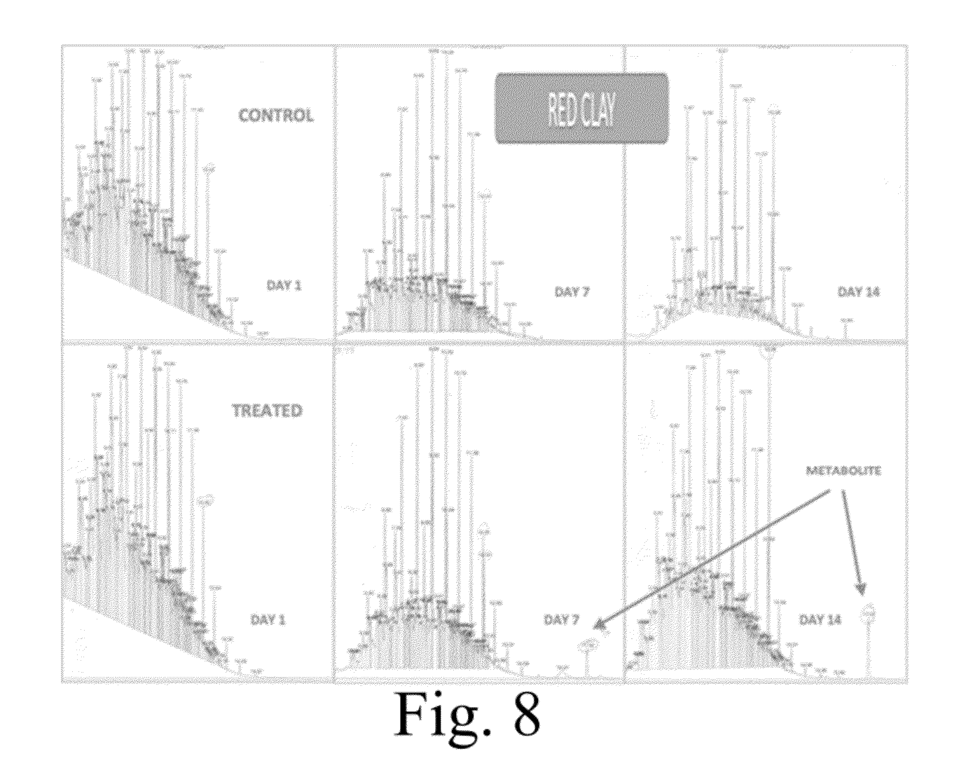

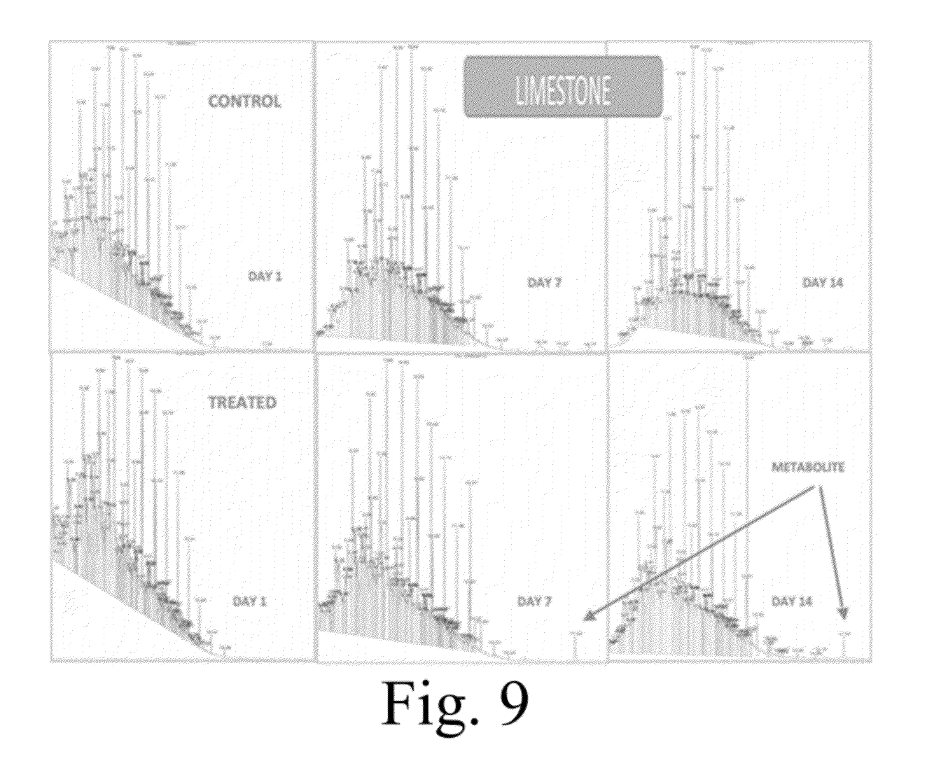

Each individual spike in the chromatogram of FIG. 8 is a relative representation of a chemical within the sample. The top row of chromatograms represents the control group, while the bottom shows the treated group. While the control group shows the standard evaporation of the low weight, highly volatile compounds, the treated group shows significant reduction in both singular PAH level as well as overall diesel concentration, up to a 13.9% degradation. The increase in bacterial metabolite level, marked below, between days 1 and 14 signifies significant growth of bacteria within the soil. The boxed peaks represent the level of Linoleic acid in comparison to the levels of diesel. Because the levels of Linoleic acid cannot go up, the image of the chromatogram is scaled to match the new levels of peaks. The Chromatogram of FIG. 9 shows the total concentration of each chemical compound found within the sample. The increasing levels of Linoleic acid and oleic acid metabolite within the treated set of data show significant remediation up to 32%. The increasing levels of the oleic acid metabolite also show continuing and increasing reaction with the diesel.

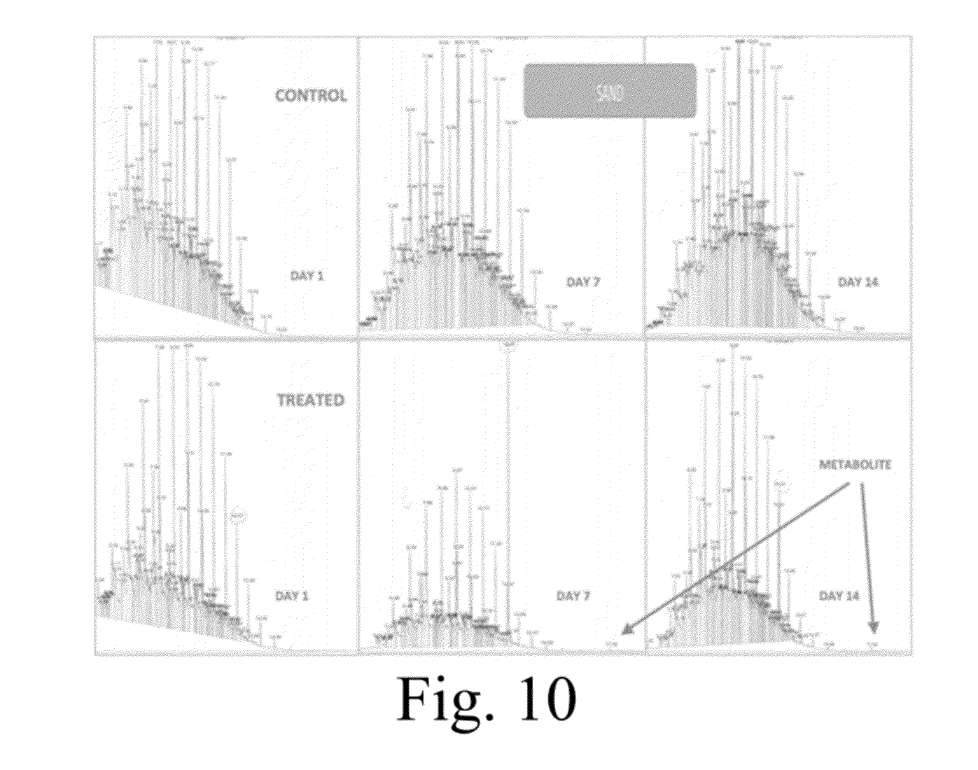

The sand chromatograms of FIG. 10 show the expectable evaporation of volatile chemicals at the far left of each chromatogram, the Linoleic acid quickly becomes the most prominent of the peaks. The metabolite on the other hand remains relatively low in comparison to other soil types. This could be attributed to lower temperatures recorded in sand during the testing period, retarding the activity of the R. rhodochrous. Despite this sand resulted in being the most successful, with a final remediation of 53%.

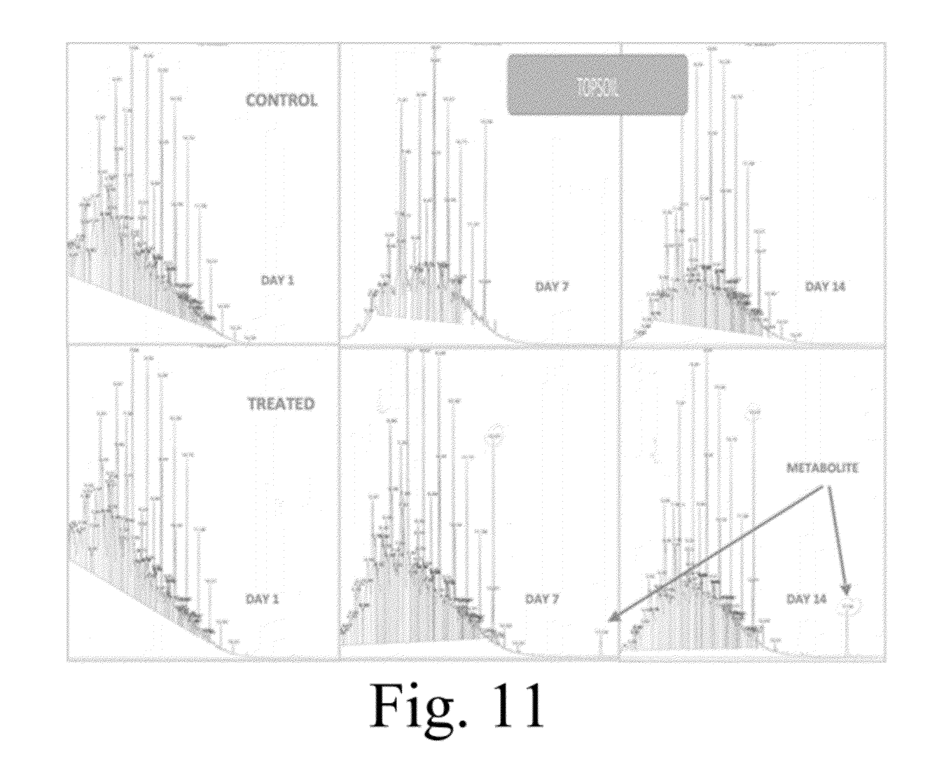

The top soil samples shown in FIG. 11 illustrate a decrease in diesel concentration consistent with that of the other soil types. Topsoil had a final remediation of 22.7% of the diesel. Topsoil is unique in the rate in which the R. rhodochrous produces oleic acid, metabolizes, and reproduces. The oleic acid metabolite becomes far more apparent than most other soil types.

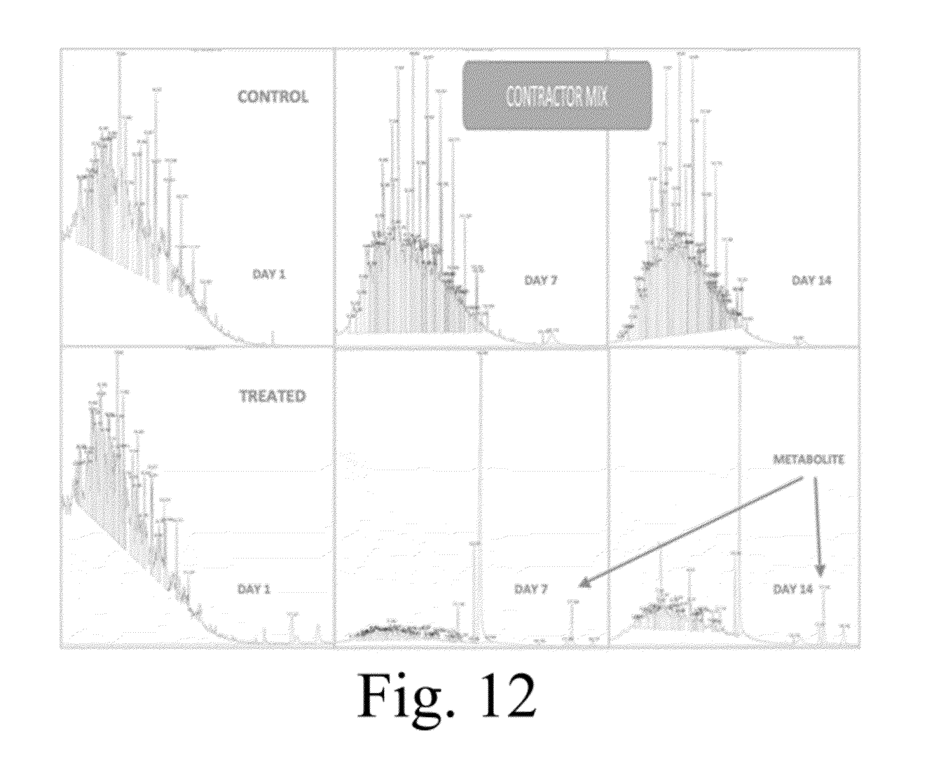

The contractor mix chromatograms shown in FIG. 12 represent an abnormality between the other soil samples. It has an extremely large initial drop in diesel concentration; there is a small increase on the 14^{th} day's diesel concentration. This is mostly attributed to human error as well as mass spectrometer error. Despite this, the contractor mix maintained a final remediation of 40%. The Linoleic acid and the R. rhodochrous metabolite continue to be very outstanding compared to other peaks.

Red Clay and Topsoil, while being the best for bacterial growth, had the lowest remediation over the two weeks, 13.9% for Red Clay and 22.4% for Topsoil. These rates may be attributed to the sorption coefficient of the soil, which causes the mixture to permeate the soil more slowly. The highest remediation rate could be found in sand with a 53% total remediation into water and methyl esters. The presence of metabolite throughout all bioremediated samples is a sign of continuing bacterial survival and consumption of PAH and other hydrocarbons. The mixture shows promise not only as a PAH remediator, but as a remediation tool for oil, diesel, and similar environmental threats based on these results.

In an alternative embodiment of the present invention, the PAH or diesel contaminated soil is replaced by PAH or diesel contaminated water or salt water. The present invention is adaptable to cleaning soil and contaminated fresh water such as lakes and streams as well as salt water lakes, oceans, or even brackish water areas.

In yet another embodiment, the present invention may be practiced in a container where the contaminated soil or water is process or stored. Although the invention, as first discovered and tested was directed to open field or open water locations, it is feasible, as testing has shown that contaminated soil or water can be gathered up and stored in containers from contaminated locations or used in contaminated locations that provide for retention or storage of contaminated spills, accidents, or production by-products.

Whereas particular embodiments of this invention have been described above for purposes of illustration, it will be evident to those skilled in the art that numerous variations of the details of the present invention may be made without departing from the invention as defined in the appended claims.

Furthermore, other areas of art may benefit from this method and adjustments to the design are anticipated. Thus, the scope of the invention should be determined by the appended claims and their legal equivalents, rather than by the examples given.

The embodiments of the invention in which an exclusive property or privilege is claimed are defined as follows:

1. A method of degrading a contaminate in soil or water, the method comprising the steps of:
maintaining a bioremediation mixture comprising a contaminated soil, at least one natural microorganism source, and at least one oxidizable lipid source for a period of time sufficient to convert at least a portion of the diesel-contaminated soil into reduced derivative compounds to give a bioremediation product; and
maintaining the bioremediation mixture under conditions for a period of time sufficient to produce a remediated soil product in which at least some of the reduced derivatives compounds have been converted into water and methyl esters.

2. The method of claim 1, wherein the natural microorganism source is R. rhodochrous bacteria.

3. The method of claim 2, wherein the lipid source is Linoleic acid.

4. The process of claim 3, wherein the remediated soil is a PAH contaminated soil.

5. The process of claim 3, wherein the remediated water is a PAH contaminated salt water.

6. The process of claim 3, wherein the remediated soil is diesel-contaminated soil.

7. The process of claim 3, wherein the remediated water is diesel-contaminated salt water.

8. The process of claim 1, wherein the weight of contaminants in the remediated soil is at least 50% less than the weight of the contaminants found in the contaminated soil.

9. The process of claim 1, wherein the bioremediation mixture includes from about 5% to about 50% by weight of the natural microorganism source.

10. The process of claim 1, wherein the bioremediation mixture includes from about 5% to about 50% by weight of the lipid source.

11. The process of claim 1, wherein the bioremediation occurs in a container.

12. The process of claim 1, wherein the percent of oleic acid in the contaminated soil increases over a period of time sufficient to produce a remediated soil product

13. A material for bioremediation against PAHs in soil or water comprising of a mixture of:
at least one natural microorganism source; and
at least one oxidizable lipid source.

14. The material of claim 11, wherein the natural microorganism source is R. rhodochrous bacteria.

15. The material of claim 11, wherein the lipid source is Linoleic acid.

16. The material of claim 11, wherein the bioremediation mixture includes from about 5% to about 50% by weight of the natural microorganism source.

17. The material of claim 11, wherein the bioremediation mixture includes from about 5% to about 50% by weight of the lipid source.

18. The material of claim 1, producing water as well as methyl esters as the result of a converting PAHs located in contaminated soil.

19. A material for bioremediation of soil or water, consisting of a mixture of
at least one natural microorganism source wherein the natural microorganism source is R. rhodochrous bacteria; and
at least one oxidizable lipid source wherein the lipid source is Linoleic acid.

20. The material for bioremediation of soil or water of claim 1, wherein the ratio of the Linoleic acid to the R. rhodochrous bacteria is three parts Linoleic acid to one part R. rhodochrous bacteria.
